(19) United States
(12) Patent Application Publication
(10) Pub. No.: US 2021/0369635 A1
(43) Pub. Date: Dec. 2, 2021

(54) NOVEL METHODS OF VACCINATION USING ICOSAHEDRAL PHAGE

(71) Applicant: Adaptive Phage Therapeutics, Inc., Gaithersburg, MD (US)

(72) Inventor: Carl Merril, Bethesda, MD (US)

(73) Assignee: Adaptive Phage Therapeutics, Inc., Gaithersburg, MD (US)

(21) Appl. No.: 16/767,737

(22) PCT Filed: Nov. 28, 2018
(86) PCT No.: PCT/US18/62884

§ 371 Date: May 28, 2020

Related U.S. Application Data
(60) Provisional application No. 62/592,104, filed on Nov. 29, 2017.

(51) Int. Cl. A61K 9/70; A61K 39/12; A61K 39/155; A61K 39/00; A61K 39/245

(52) U.S. Cl. CPC A61K 9/7023; A61K 2039/545; A61K 39/155; A61K 39/001186; A61K 39/00117; A61K 39/245; A61K 39/001184; A61K 39/001151; A61K 39/001189; A61K 39/001191; A61K 39/001166; A61K 39/001156; A61K 2039/54; A61K 2039/5252; A61K 2039/5256; A61K 39/12

(57) ABSTRACT

A transdermal membrane comprising a non-infectious icosahedral phage vaccine displaying an antigen is described wherein the membrane is stable at room temperature for greater than 3 months and uses thereof to vaccinate a subject against the antigen.

### BACKGROUND OF THE INVENTION

In the following discussion, certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Transdermal delivery offers compelling opportunities to improve vaccine administration. Although vaccines are typically macromolecules, viral particles, or other large supramolecular constructs, their small (microgram) doses facilitate the possibility of transdermal delivery. Vaccine delivery via the skin is even more attractive because it targets the potent epidermal Langerhans and dermal dendritic cells that may generate a strong immune response at much lower doses than deeper injection (1). The most successful vaccine of all time—the smallpox vaccine, which eradicated the disease worldwide—was administered via the skin with the aid of a small needle device to breach the stratum corneum barrier. Although effective, this approach did not provide good control over delivery, which has motivated development of new delivery methods.

Elimination of the need for hypodermic needles further motivates transdermal vaccine development (2). In a world where needle reuse kills at least 1.3 million people per year from hepatitis B and AIDS (3), needle-free, patch-based vaccination could have a large impact. In addition, the possibility of administering vaccine patches by minimally trained personnel or patients themselves could not only facilitate compliance with routine, seasonal and pandemic vaccination needs, but could also expedite vaccination campaigns in developing countries where medical personnel are in short supply. Effective vaccination via the skin may be achieved by increasing skin permeability to the vaccine using the methods as described in the art. Some of the physical enhancement methods have been shown to have additional adjuvant effects that increase immune response further (4,5). The immune response can also be heightened by adding chemical adjuvants (1).

Excitement about this approach is exemplified by completion of phase 3 clinical trials and submission for registration in Europe by Sanofi Pasteur (Paris) and Becton Dickinson (Franklin Lakes, N.J., USA) for their microneedle-based influenza vaccine; major investments in Iomai for their transdermal vaccine patch portfolio; and a growing number of academic and industry laboratories engaging in this field of research. For a sense of perspective, one of the first vaccine used for human disease, the smallpox vaccine (using suspended material from a cowpox lesion), was in essence, a transdermal vaccination, performed by scratching or abrading the skin, initially with a bone fragment in the presence of a drop of suspended cowpox material. This vaccine, pioneered and widely communicated by Edward Jenner in the 1790's eventually led to the global elimination of smallpox in 1980.

In US Patent Application 2007/027167, a recombinant functional phage expressing an antigen along with an immunogenic enhancer molecule was used to vaccinate a patient. The phage was processed by professional antigen presenting cells (APCs), such as dendritic cells, where efficient expression of the vaccine genes occurs (Clark and March, 2004). This approach relies upon mammalian promotors and a functional phage DNA/genome for efficient expression of the pathogen and enhancer in the target cells—for vaccines, this would generally be the immune dendritic cells. In this approach, once the functional phage are delivered to the host organism, e.g., by injection, the phage is taken up by the mammalian professional antigen presenting cells where the antigen is expressed and displayed on its surface to stimulate an immune response.

Other similar approaches utilize a delivery system having a lambda (phage) construct, with C-terminal fusions between the gpD external virion protein and the IgG-binding domains of staphylococcal protein A and streptococcal protein G. Purified A phage with both fusion types were used in conjunction with antibodies specific for common dendritic cell receptors to target human and murine dendritic cells in vitro. In this example, the fusion product with a coat protein was being used to target and stimulate the immune dendritic cells, while the vaccine vector was packaged separately in the phage genome with a mammalian promoter for expression once the phage was taken up by the dendritic cells.

Thus, traditional approaches of phage based vaccines rely on recombinant expression of an antigen and promoter (recombinantly inserted into the phage genome) when the phage is taken up by an immune cell such as a dendritic cell. Once in the dendritic cell, the mammalian promoter directs expression of the antigen in the dendritic cells. The expressed antigens are processed and displayed on the surface of dendritic cells for activation of the immune response, including T-cell activation and production of antibodies. However, these approaches require that the phage be viable in order to trigger an immune response. Such viable phage formulations require that the phage stocks be stored cold (e.g., in refrigerators or freezers) and have a short half-life when kept at room temperature. Moreover, use of viable phage based vaccines have increased regulatory hurdles due to concerns by regulatory agencies of infections or off-target side-effects. Thus, there is a need for improved methods of vaccination.

### SUMMARY OF THE INVENTION

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject.

According to embodiments of the present invention, inactive icosahedral phage displaying vaccine epitopes (antigens) are used for vaccination. Such compositions are then dried on transdermal membranes (or any other suitable transdermal delivery system) and stored at room temperature for extended periods of time for subsequent use.

Unlike other approaches, the present invention utilizes icosahedral phage machinery to display an antigen (vaccine epitope) on its surface of the phage head, e.g., as a fusion product between the antigen and an icosahedral phage coat protein. By attaching the antigen to an icosahedral phage coat protein, the antigen is localized to the icosahedral phage surface, where is it readily accessible for processing by immune dendritic cells (DCs). The DCs will react to this icosahedral phage coat protein antigen construct without relying on internal expression of the antigen in the dendritic cells.

The invention relates to a transdermal membrane comprising a non-infectious icosahedral phage vaccine displaying an antigen, wherein the membrane is stable at room temperature for greater than 3 months. In preferred embodiments, the non-infectious icosahedral phage vaccine is either heat inactivated or inactivated using UV light. The non-infectious icosahedral phage vaccine can be inactivated prior to application onto the membrane or inactivated after application onto the membrane.

In further embodiments, the membrane is stable at room temperature for greater than. 6 months, 9 months, 12 months, 18 months, 24 months, 30 months or 36 months. The membrane can also be capable of abrading the skin surface.

In preferred embodiments, the antigen is displayed as a fusion protein with an icosahedral phage coat protein. Examples of such icosahedral phage coat protein include D major coat protein of lambda phage or other lambdoid phage.

In preferred embodiments, the antigen is selected from: (a) a bacterium or a cancer antigen; (b) a cancer antigen selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA125, CA15-3 (CA 27.29 \BCAA), CA195, CA 242, CA-50, CAM43, CD68 \KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, mesothelin or any combination thereof; (c) a bacterium selected from a Risk Group IV bacterium; (d) a Risk Group IV bacterium selected from Arenaviruses (e.g., Guanarito virus, Lassa virus, Junin virus, Machupo virus, Sabia, Bunyaviruses (Nairovirus): Crimean-Congo hemorrhagic fever virus), Filoviruses (e.g., Ebola virus and Marburg virus), Flaviruses (Togaviruses)(e.g., Group B Arboviruses: Tick-borne encephalitis virus complex including Absetterov, Central European encephalitis, Hanzalova, Hypr, Kumlinge, Kyasanur Forest disease, Omsk hemorrhagic fever, and Russian spring-summer encephalitis viruses), Herpesviruses (alpha) (Herpesvirus simiae (Herpes B or Monkey B virus)), Paramyxoviruses (e.g., Equine morbillivirus (Hendra virus)); Hemorrhagic fever agents and viruses as yet undefined, or any combination thereof. An example of a Hemorrhagic fever agent includes but is not limited to Ebolavirus, Bundibugyo ebolavirus, Reston ebolavirus, Sudan ebolavirus, Taï Forest ebolavirus (originally Côte d'Ivoire ebolavirus), Zaire ebolavirus, or any combination thereof.

In further preferred embodiments, more than one antigen is displayed. In other preferred embodiments, the icosahedral phage vaccine further comprises a polynucleotide encoding a second antigen operably associated with a promoter capable of being expressed in a mammalian cell. Examples of such promoters are well known in the art.

In preferred embodiments, the second antigen is derived from the same protein as the displayed antigen or alternatively, the second antigen is different from the displayed antigen. In further preferred embodiments, the polynucleotide is inserted into the icosahedral phage vaccine genome and/or encodes for multiple antigens. In further preferred embodiments, the second antigen is selected from: (a) a bacterium or a cancer antigen; (b) a cancer antigen selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A2, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA19-9, CA 72-4, CAM 17.1, NuMa, K-ras, alpha.-fetoprotein, 13HCG, BCA225, BTAA, CA 125, CA 15-3 (CA 27.29 \BCAA), CA195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, mesothelin or any combination thereof; (c) a bacterium selected from a Risk Group IV bacterium; (d) a Risk Group IV bacterium selected from Arenaviruses (e.g., Guanarito virus, Lassa virus, Junin virus, Machupo virus, Sabia, Bunyaviruses (Nairovirus): Crimean-Congo hemorrhagic fever virus), Filoviruses (e.g., Ebola virus and Marburg virus), Flaviruses (Togaviruses)(e.g., Group B Arboviruses: Tick-borne encephalitis virus complex including Absetterov, Central European encephalitis, Hanzalova, Hypr, Kumlinge, Kyasanur Forest disease, Omsk hemorrhagic fever, and Russian spring-summer encephalitis viruses), Herpesviruses (alpha) (Herpesvirus simiae (Herpes B or Monkey B virus)), Paramyxoviruses (e.g., Equine morbillivirus (Hendra virus)); Hemorrhagic fever agents and viruses as yet undefined, or any combination thereof. An example of a Hemorrhagic fever agent includes but is not limited to Ebolavirus, Bundibugyo ebolavirus, Reston ebolavirus, Sudan ebolavirus, Taï Forest ebolavirus (originally Côte d'Ivoire ebolavirus), Zaire ebolavirus, or any combination thereof.

The invention also relates to a method of vaccinating a subject wherein the method comprises contacting the skin of the subject with any of the membrane as described herein. The subject vaccinated can be preferably a human subject. The subject can also be a non-human subject. In preferred embodiments, the subject is vaccinated against a cancer or a bacterial infection.

Examples of cancers that can be treated using the membranes as described herein include, but are not limited to: a sarcoma, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, stomach cancer, multiple myeloma, cerebral cancer, adenocarcinoma, pancreatic cancer, or pancreatic ductal adenocarcinoma.

Examples of bacterial infections that can be treated as described herein include infections caused by a Risk Group IV bacterium, including hemorrhagic infections. The method of vaccinating a subject can be (a) performed prophylactically; and/or (b) repeated to boost the immune response; and/or (c) part of a prime-boost protocol.

### BRIEF DESCRIPTION OF THE DRAWINGS

FIG. 1A and FIG. 1B illustrates two ways in which an icosahedral phage vaccine as described herein can be constructed.

FIG. 2 illustrates exemplified constructs that can be used to display the antigen on the icosahedral phage head.

FIGS. 3-5 illustrate exemplified constructs that can be used to integrate the antigen in the icosahedral phage vaccine genome

### DETAILED DESCRIPTION OF THE INVENTION

### Definitions

The following definitions are provided for specific terms are used in the following written description.

Unless defined otherwise, all technical and scientific terms used herein have the same meaning as commonly understood by those of ordinary skill in the art, such as in the arts of peptide chemistry, immunology, cell culture and icosahedral phage display, nucleic acid chemistry and biochemistry. Standard techniques are used for molecular biology, genetic and biochemical methods (see Sambrook et al., Molecular Cloning: A Laboratory Manual, 3^{rd} ed., 2001, Cold Spring Harbor Laboratory Press, Cold Spring Harbor, N.Y.; Ausubel et al., Short Protocols in Molecular Biology (1999) 4th ed., John Wiley & Sons, Inc.), which are incorporated herein by reference.

As used in the specification and claims, the singular form “a”, “an” and “the” include plural references unless the context clearly dictates otherwise. For example, the term “a cell” includes a plurality of cells, including mixtures thereof. The term “a nucleic acid molecule” includes a plurality of nucleic acid molecules. “An antigen” can mean at least one antigen, as well as a plurality of antigens, i.e., more than one antigen. As understood by one of skill in the art, the term “icosahedral phage” can be used to refer to a single icosahedral phage or more than one icosahedral phage.

The present invention can “comprise” (open ended) or “consist essentially of” the components of the present invention as well as other ingredients or elements described herein. As used herein, “comprising” means the elements recited, or their equivalent in structure or function, plus any other element or elements which are not recited. The terms “having” and “including” are also to be construed as open ended unless the context suggests otherwise. As used herein, “consisting essentially of” means that the invention may include ingredients in addition to those recited in the claim, but only if the additional ingredients do not materially alter the basic and novel characteristics of the claimed invention.

As used herein, a “subject” is a vertebrate, preferably a mammal, more preferably a human. Mammals include, but are not limited to, murines, simians, humans, farm animals, sport animals, and pets. In other preferred embodiments, the “subject” is a rodent (e.g., a guinea pig, a hamster, a rat, a mouse), murine (e.g., a mouse), canine (e.g., a dog), feline (e.g., a cat), equine (e.g., a horse), a primate, simian (e.g., a monkey or ape), a monkey (e.g., marmoset, baboon), or an ape (e.g., gorilla, chimpanzee, orangutan, gibbon). In other embodiments, non-human mammals, especially mammals that are conventionally used as models for demonstrating therapeutic efficacy in humans (e.g., murine, primate, porcine, canine, or rabbit animals) may be employed. Preferably, a “subject” encompasses any organisms, e.g., any animal or human, that are in need of a vaccine.

As understood herein, an “effective amount” of a pharmaceutical composition of the instant invention refers to an amount of the composition suitable to elicit a therapeutically beneficial response in the subject, e.g., generating an immune response against the antigen presented in the vaccine. Such response may include e.g., preventing, ameliorating, treating, inhibiting, and/or reducing one of more diseases associated with the antigen.

The term “dose” or “dosage” as used herein refers to physically discrete units suitable for administration to a subject, each dosage containing a predetermined quantity of the active pharmaceutical ingredient calculated to produce a desired response.

The term “about” or “approximately” means within an acceptable range for the particular value as determined by one of ordinary skill in the art, which will depend in part on how the value is measured or determined, e.g., the limitations of the measurement system. For example, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5 fold, and more preferably within 2 fold, of a value. Unless otherwise stated, the term ‘about’ means within an acceptable error range for the particular value, such as ±1-20%, preferably ±1-10% and more preferably ±1-5%. In even further embodiments, “about” should be understood to mean +/−5%.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

All ranges recited herein include the endpoints, including those that recite a range “between” two values. Terms such as “about,” “generally,” “substantially,” “approximately” and the like are to be construed as modifying a term or value such that it is not an absolute, but does not read on the prior art. Such terms will be defined by the circumstances and the terms that they modify as those terms are understood by those of skill in the art. This includes, at very least, the degree of expected experimental error, technique error and instrument error for a given technique used to measure a value.

Where used herein, the term “and/or” when used in a list of two or more items means that any one of the listed characteristics can be present, or any combination of two or more of the listed characteristics can be present. For example, if a composition of the instant invention is described as containing characteristics A, B, and/or C, the composition can contain A feature alone; B alone; C alone; A and B in combination; A and C in combination; B and C in combination; or A, B, and C in combination.

As used herein, “non-infectious icosahedral phage” refer to icosahedral phage that have either naturally lost the ability to be infectious or icosahedral phage that have lost the ability to be infectious in vitro, such as by “ultraviolet irradiated”, “heat-killed” or “heat-inactivated.” A “non-infectious icosahedral phage” can also include icosahedral phage with an inactivated genome such that it can no longer infect a bacterium or other organism, but yet still presents an antigen wherein the antigen is fused to an icosahedral phage coat protein. Inactive icosahedral phage and non-infection icosahedral phage are used interchangeably throughout the specification.

As used herein “icosahedral phage” means a phage having an icosahedral shaped head. The structure of such phage heads allow for maximal presentation of an antigen when fused to an icosahedral phage coat protein. Examples of such icosahedral phage, include, but are not limited to phage classified as lambdoid phage, myoviridae (e.g., coliphage, T2, T4, or T6); microviridae (e.g., φX174); cystoviridae (e.g., phage φ6) styloviridea (e.g., T₁ or T₅); levivirdiae (e.g., phage MS₂ and Qβ); pedoviridae, corticoviridae (pseudomonas phage MP₂); or tectiviridae (e.g., PRD₁). In preferred embodiments, Lambda phage are used to generate the non-infectious icosahedral phage vaccine as described herein.

As used herein, “transdermal membrane” refers to a membrane that is applied to the surface of the skin, or implanted just beneath the surface of the skin. According to present invention embodiments, an icosahedral phage expressing a fusion protein comprising the antigen and an icosahedral phage coat protein is applied to the membrane and dried. The transdermal membrane delivers the antigen to the host organism to trigger an immune response. Transdermal membranes are manufactured by companies such as 3M (6).

As used herein, “transdermal administration” refers to using a transdermal membrane for administration of the antigen.

As used herein, “antigen” or “epitope” or “vaccine epitope” refers to an amino acid peptide of a pathogen of interest. Once administered to a host organism, the antigen will trigger an immune response in the host. Examples of pathogens from which antigens may be derived include are known in the art and described herein.

As used herein, “stable” refers to being in a form that is not subject to degradation.

As used herein, “fusion protein” refers to a protein that comprises all or at least part of two naturally occurring proteins. It will be appreciated that naturally occurring proteins are not considered to constitute a “fusion protein” in accordance with the present invention. Thus, a protein that essentially consists of a sequence from a single naturally occurring protein (or a variant thereof) and without the introduction of amino acid sequences from a second protein, does not constitute a fusion protein according to present invention embodiments. According to present invention embodiments, the fusion protein comprises at least one antigen fused to an icosahedral phage coat protein.

As used herein, “bacterial host” refers to a host organism used for propagation of the icosahedral phage, which has been modified to express the fusion protein and to be used as the non-infectious icosahedral phage vaccine.

As used herein, “icosahedral phage coat protein” refers to a protein that forms the viral envelop/capsid of an icosahedral phage. Examples of icosahedral phage coat protein that can be used include but are not limited to the D major coat protein found on Lambda phage or equivalent proteins found in other lambdoid phage. In preferred embodiments, the D major coat protein is used to generate the fusion protein as described herein as there are 405 copies of the D major coat protein on each Lambda head, giving a much higher dose of antigen as compared to when an antigen is displayed on a filamentous phage. Although methods are known in the art for displaying antigens by fusions proteins with filamentous phage coat proteins, these methods have not proved effective. See, Henry et al., “Beyond Phage Display: Non-Traditional Applications of the Filamentous Bacteriophage as a Vaccine Carrier, Therapeutic Biologic, and Bioconjugation Scaffold,” Front Microbiol 6:755 (2015) (herein incorporated by reference in its entirety).

As used herein, “vaccination” refers to administration of an antigen to trigger an adaptive immune response to the antigen.

As used herein, a “CpG site” refers to a region of DNA where a cytosine nucleotide is followed by a guanine nucleotide.

The term “pharmaceutically acceptable” as used herein pertains to compounds, materials, compositions, and/or dosage forms which are, within the scope of sound medical judgment, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

The term “therapeutically-effective amount” as used herein, pertains to that amount of an active compound, or a combination, material, antigen, composition or dosage form comprising an active compound, which is effective for producing some desired therapeutic effect, commensurate with a reasonable benefit/risk ratio.

### Inactivated Icosahedral Phage

In one embodiment, an icosahedral phage genome is modified to express a fusion protein comprising at least one antigen and an icosahedral phage coat protein, while the icosahedral phage propagates in a bacterial host, prior to its use as a vaccine. Any molecule in the icosahedral phage genome (e.g., a coat protein, or any other protein) localized to the surface or exterior of the bacterioicosahedral phage can be genetically fused to an antigen/epitope for use as an icosahedral phage vaccine as described herein. Such expression of an antigen on the surface of an icosahedral phage can be produced according to icosahedral phage display techniques as well known in the art and described herein.

For example, the expressed fusion protein comprises at least one antigen and icosahedral phage coat protein. The coat protein is localized to the exterior of the icosahedral phage, where it is presented for interaction with an antigen presenting cell. However, unlike what was described previously, the icosahedral phage used in the present invention is inactive as the fusion protein (antigen-coat protein) acts as the vaccine epitope and does not require host processing in immune cells.

Additionally, the icosahedral phage vaccine vector can also carry a DNA construct integrated into the genome containing a mammalian promotor and a gene for a potential vaccine antigen that is to be expressed by the immune dendritic cells after being taken up by the dendritic cells during immunization. Again, viable icosahedral phage are not required or even advantageous for either of these delivery systems. In preferred embodiments both (a) the display of an antigen fused to an icosahedral phage coat protein on the surface of the icosahedral phage head and (b) a DNA construct integrated into the icosahedral phage genome containing a mammalian promotor and a gene for a potential vaccine antigen that is to be expressed by the immune dendritic cells when the icosahedral phage are taken up by the dendritic cells during immunization are used as a vaccine as described herein.

Thus, present invention provides for a method and system of presenting an antigen as part of a fusion protein to be used as a vaccine in a mammal, where the fusion protein comprises the antigen and an icosahedral phage coat protein. In preferred embodiments this approached is combined with the delivery on a polynucleotide comprising an antigen operably associated with a mammalian promoter capable of being expressed by the host's immune cells after being taken up by dendritic cells during immunization. These approaches have a number of advantages, including simplification of manufacturing a vaccine, and most importantly, an improved safety profile and stability at room temperature.

According to embodiments of the present invention, the icosahedral phage genome is inactivated. (rendered nonfunctional) by heat or UV light and the fusion product (coat protein fused to the vaccine epitope/antigen), prior to administration, and can still act as a vaccine and induce an immune response. Most proteins are more resistant to UV and heat damage than genomic DNA. Therefore, icosahedral phage vaccine vectors carrying the vaccine epitope fused to an icosahedral phage coat protein, could be made, and inserted into an icosahedral phage for expression. The icosahedral phage could then be replicated, isolated, placed on a “transdermal membrane”, and dried and stored for later use.

Additionally, once an epitope or antigen stimulating peptide is made into a fusion product with the coat protein of an icosahedral phage, such as Lambda phage, the construct as a vaccine, is temperature stable, and no longer requires a functional icosahedral phage genome.

Icosahedral phage can be inactivated by UV light, by drying on a membrane, or any other suitable technique for inactivating icosahedral phage genomes. Once inactivated, the icosahedral phage can be stored at room temperature for years, provided that the icosahedral phage are maintained in a dry environment.

Additional aspects of the present invention include displaying more than one fusion protein on an icosahedral phage and/or expression of multiple antigens by the mammalian promoter operably associated on the inserted polynucleotide construct. Still further embodiments including administering multiple types of icosahedral phages, each type of icosahedral phage displaying a different epitope/peptide antigen by application to a single membrane. Thus, a single application of a single membrane can be used for multivalent or multiple vaccines in a single application.

The use of a bacterial virus, including but not limited to lambdan icosahedral phage, eliminates the need for an external adjuvant because the icosahedral phage are grown in bacteria and their DNA are not methylated in the same manner as human or animal DNA, and in particular at CpG sites. Thus, the immune system recognizes the icosahedral phage DNA and attached fusion protein as foreign, and mounts an immune response against the antigen (presented as the fusion construct).

This novel combination of inactive icosahedral phage with antigen fused to a coat protein (and/or expression of multiple antigens by the operably associated mammalian promoter on the inserted polynucleotide construct) and transdermal delivery system represents a novel technology having a variety of features. This technology is easy and inexpensive to design, and produces genetically engineered icosahedral phage that display a vaccine epitope as a fusion product on its surface (due to the coat protein). Such constructs can be dried on membranes designed for transdermal delivery or for subdermal implantation, and do not need specialized storage facilities. The vaccine remains stable and may be stored for years. More than one vaccine construct can be applied on a single membrane so that multiple vaccinations can be performed at one time.

Such vaccine systems can open a whole new array of vaccine applications. For example, such vaccine-membrane systems could be stored at room temperature for years without degradation. They can also be dispensed by prescription without the need for expert administration (e.g., more or less in the matter of a Band-Aid). In addition, multiple different icosahedral phage based vaccines could be applied to a single membrane so that with one application, a number of immunizations could be provided to patients.

Given the relative ease of synthesizing such icosahedral phage vaccines and the low-cost of producing them (e.g., vaccines can be produced in simple fermenters or even in flasks using relatively inexpensive growth media), the vaccines could be produced in a fraction of the amount of time that it currently takes to produce most traditionally manufactured vaccines, such as influenza vaccines. It should be possible to produce icosahedral phage display based vaccines within a week or two after the structure of an antigen found in a pathogen is determined.

Such vaccines could revolutionize our ability to prevent diseases in countries lacking in financial resources. In addition, with a little training and a relatively small investment, such countries could produce their own vaccines locally.

The robust nature of such vaccines would also permit specialized vaccines to be developed and produced for protection against biological warfare to be stockpiled at little cost for long periods of time.

### Transdermal Delivery

Transdermal delivery provides an attractive option for the delivery of vaccines and other therapeutic products. Transdermal delivery involves the application of the antigen to the surface of the skin, where the antigen passively diffuses through the surface of the skin, or alternatively, the implantation of the antigen within the skin, preferably just below an outer layer of the skin. Transdermal delivery systems are available and such systems can be optimized to interface with the present invention (6).

For delivery to the surface of the skin, the uppermost layer of the skin, the stratum corneum, is known to be a barrier to the delivery of water soluble compounds, e.g. peptides, vaccines, etc. In embodiments of the invention, the inactive icosahedral phage displaying at least one antigen will be provided as a formulation (a liquid, a solution, an oil, etc.) designed to transport the antigen across this barrier.

In other embodiments, microporation technologies may place discrete holes into the skin applying a drug-delivery patch. Microporation technologies may use thermal energy, radiofrequency or mechanical disruption to create channels in the stratum corneum for delivery of the drug. When followed by application of a transdermal patch, these micropore technologies enhance delivery of hydrophilic drugs and peptides, e.g., such as vaccines and inactivated icosahedral phage particles.

In still other embodiments, the skin may be pre-treated with an abrasive substance to disrupt the stratum cornuem. After removal of this abrasive strip, a patch containing the vaccine is applied over the treatment site.

In still other embodiments, the icosahedral phage vaccine as described herein may be coated into microneedles that are embedded into the skin, where the antigen is released into the epidermis or dermis. If the membrane contains micro or nano-needle like structures, such a membrane can be merely applied to a “hairless” region of the skin of an individual or animal to vaccinate the animal for the selected epitope or peptide antigen.

In other embodiments, the transdermal delivery system may be applied using power and jet injectors or iontophoric devices.

In still other embodiments, transdermal delivery systems may be in the form of an adhesive patch that is transiently adhered to the skin. The patch contains the inactivated icosahedral phage presenting the fusion protein construct to be used as a vaccine. Common materials for the adhesive patch include, but are not limited to, a simple adhesive “Band-Aid” like product to a microplastic product having protrusions that abrade the skin. The patch may be transiently implanted below the skin to deliver the fusion protein to the dermis and epidermis. For example, the patch may be temporarily implanted below the surface of the skin, e.g., for 1 minute, 2 minutes, 5 minutes, 10 minutes, 15 minutes, 30 minutes and so forth before removal in order to deliver the antigen.

Once the inactivated icosahedral phage displaying the antigen via the fusion protein, and/or expression of at least one antigen by the operably associated mammalian promoter encoded by the polynucleotide construct inserted into the icosahedral phage genome, passes through the stratum corneum to reach the epidermis and dermis, the antigen may be taken up by specialized cells of the immune system, e.g., Langerhans cells and dermal dendritic cells, where the antigen is processed and presented to T-cells to initiate an immune response.

Transdermal administration is thought to trigger immune responses more quickly and with greater intensity than vaccines administered using intramuscular injection. It is thought that Langerhans cells and dermal dendritic cells, which are transported directly to the secondary lymph nodes via draining lymphatic capillaries are large responsible for this effect. Lymphatic capillaries act as a conduit of the immune system, providing a pathway for the migration of T- and B- cells as well as a conduit for the trafficking of antigen presenting cells to the lymph nodes. Proteins, macromolecules, vaccines and other biologics are cleared from interstitial space using the transport system of the lymphatic capillaries.

Human clinical trials have been shown intradermal delivery to be an effective mode of vaccine delivery. For instance, Tuft et al. showed that an antibody response roughly equivalent to intramuscular or subcutaneous delivery could be generated using intradermal delivery with reduced amounts of antigen.

In some embodiments, the concentration of the icosahedral phage vaccine delivered with a transdermal delivery system is about 10, 50, 100, 150 or 200 million icosahedral phage. In further preferred embodiments, the concentration of the icosahedral phage vaccine delivered with a transdermal delivery system is between about 10-50 million icosahedral phage, between 50-100 million icosahedral phage, between 75-125 million icosahedral phage, between 100-125 million icosahedral phage, between 100-150 million icosahedral phage, between 150-200 million icosahedral phage, between 100-200 million icosahedral phage, between 75-150 million icosahedral phage, or between 50-250 million icosahedral phage. In other preferred embodiments, the concentration of the antigen delivered with transdermal administration is 1/2, 1/3, 1/4, 1/5, 1/6, 1/7, 1/8, 1/9, 1/10, 1/20 or less as compared to the amount needed to affect a comparable immune response in intramuscular or subcutaneous delivery.

### Antigens

Any antigen can be used as described herein.

For example, tumor antigens that can be used according to the present invention include, but are not limited to MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA19-9, CA 72-4, CAM 17.1, NuMa, K-ras, alpha.-fetoprotein, 13HCG, BCA225, BTAA, CA 125, CA 15-3 (CA 27.29\BCAA), CA195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, or mesothelin.

Examples of tumors that can be treated using the present invention, include, but are not limited to, sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, stomach cancer, multiple myeloma and cerebral cancer. Preferred embodiments of tumors are adenocarcinomas. In some embodiments, the cancer may be pancreatic cancer, for example pancreatic ductal adenocarcinoma.

Bacterial infections can also be treated using the transdermal vaccines as described herein. Preferred bacterial infections that can be treated as described herein include those identified as “Risk Group IV” bacteria. Members of this Risk Group include, but are not limited to Ebolavirus, Marburgvirus, and Lassavirus. In further embodiments, the bacterial infections of Risk Group IV, including but are not limited to: Arenaviruses (e.g., Guanarito virus, Lassa virus, Junin virus, Machupo virus, Sabia, Bunyaviruses (Nairovirus): Crimean-Congo hemorrhagic fever virus), Filoviruses (e.g., Ebola virus and Marburg virus), Flaviruses (Togaviruses)(e.g., Group B Arboviruses: Tick-borne encephalitis virus complex including Absetterov, Central European encephalitis, Hanzalova, Hypr, Kumlinge, Kyasanur Forest disease, Omsk hemorrhagic fever, and Russian spring-summer encephalitis viruses), Herpesviruses (alpha) (Herpesvirus simiae (Herpes B or Monkey B virus)), Paramyxoviruses (e.g., Equine morbillivirus (Hendra virus)); Hemorrhagic fever agents and viruses as yet undefined. Thus, antigens derived from a Risk Group IV bacteria can be used to generate a fusion protein to an icosahedral phage coat protein, displayed on the icosahedral phage, and then used in transdermal vaccination as described herein.

Preferred examples of bacterial infections resulting in hemorrhagic infections, such as those caused Ebolavirus, include Bundibugyo ebolavirus, Reston ebolavirus, Sudan ebolavirus, Taï Forest ebolavirus (originally Côte d'Ivoire ebolavirus), and Zaire ebolavirus. Thus, antigens derived from Ebolavirus (for example) can be used to generate a fusion protein to an icosahedral phage coat protein, displayed on the icosahedral phage, and then used in transdermal vaccination as described herein.

### Vaccine Compositions

Compositions suitable for vaccination using the icosahedral phage vaccines described herein may be prepared by admixing the inactivated icosahedral phage displaying the fusion protein with conventional excipients, i.e., pharmaceutically acceptable organic or inorganic carrier substances suitable for transdermal administration and that do not degrade the fusion protein. Preferably after immunization, such vaccines also are capable of expressing at least one antigen by the operably associated mammalian promoter encoded by the polynucleotide construct inserted into the icosahedral phage genome.

In preferred embodiments, the patient is a human. In other preferred embodiments, the “patient” or “subject suitable for treatment” may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In other embodiments, non-human mammals, especially mammals that are conventionally used as models for demonstrating therapeutic efficacy in humans (e.g. murine, primate, porcine, canine, or rabbit animals) may be employed.

A pharmaceutical composition may comprise, in addition to the vaccine, one or more pharmaceutically acceptable carriers, adjuvants, excipients, diluents, fillers, buffers, stabilizers, preservatives, lubricants, or other materials well known to those skilled in the art. Suitable materials will be sterile and pyrogen-free, with a suitable isotonicity and stability. Examples include sterile saline (e.g. 0.9% NaCl), water, dextrose, glycerol, ethanol or the like or combinations thereof. Such materials should be non-toxic and should not interfere with the efficacy of the active compound. The precise nature of the carrier or other material will depend on the route of administration, which may be transdermal or any other suitable route, as discussed below. Suitable materials will be sterile and pyrogen free, with a suitable isotonicity and stability. Examples include sterile saline (e.g. 0.9% NaCl), water, dextrose, glycerol, ethanol or the like or combinations thereof. The composition may further contain auxiliary substances such as wetting agents, emulsifying agents, pH buffering agents or the like.

Suitable carriers, excipients, etc. can be found in standard pharmaceutical texts, for example, Remington's Pharmaceutical Sciences, 18th edition, Mack Publishing Company, Easton, Pa., 1990.

Treatment may be any treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, delaying, abating or arresting one or more symptoms and/or signs of the condition or prolonging survival of a subject or patient beyond that expected in the absence of treatment.

Treatment as a prophylactic measure (i.e. prophylaxis) is also included. For example, a subject susceptible to or at risk of the occurrence or re-occurrence of cancer or some other infectious disease may be treated as described herein. Such treatment may prevent or delay the occurrence or re-occurrence of cancer or infectious disease in the subject.

It will be appreciated that appropriate dosages of the active compounds can vary from patient to patient. Determining the optimal dosage will generally involve the balancing of the level of therapeutic benefit against any risk or deleterious side effects of the administration.

Administration in vivo can be affected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician.

The vaccine compositions according to embodiments of the present invention are administered via a transdermal patch as described herein. The vaccination schedule will depend on the patient's response using physician's experience and judgement. By vaccinating the subject, an immune response is triggered through activation of immune cells such as antigen presenting cells (e.g., dendritic cells, macrophages, B-lymphocytes, etc.).

Exemplary steps for vaccination include applying inactivated icosahedral phage (about 10, 50, 100, 150 or 200 million icosahedral phage, or between about 10-50 million icosahedral phage, between 50-100 million icosahedral phage, between 75-125 million icosahedral phage, between 100-125 million icosahedral phage, between 100-150 million icosahedral phage, between 150-200 million icosahedral phage, between 100-200 million icosahedral phage, between 75-150 million icosahedral phage, or between 50-250 million icosahedral phage) to the skin. Antigen presenting cells will process the fusion protein construct, and will present the antigen or a fragment thereof from the fusion protein to T-cells to trigger immune activation. Subsequent booster vaccinations and/or a prime/boost schedule may be used as needed.

It is to be understood that the application discloses all combinations of any of the above aspects and embodiments described above with each other, unless the context demands otherwise. Similarly, the application discloses all combinations of the preferred and/or optional features either singly or together with any of the other aspects, unless the context demands otherwise.

### EXAMPLES

### Example 1

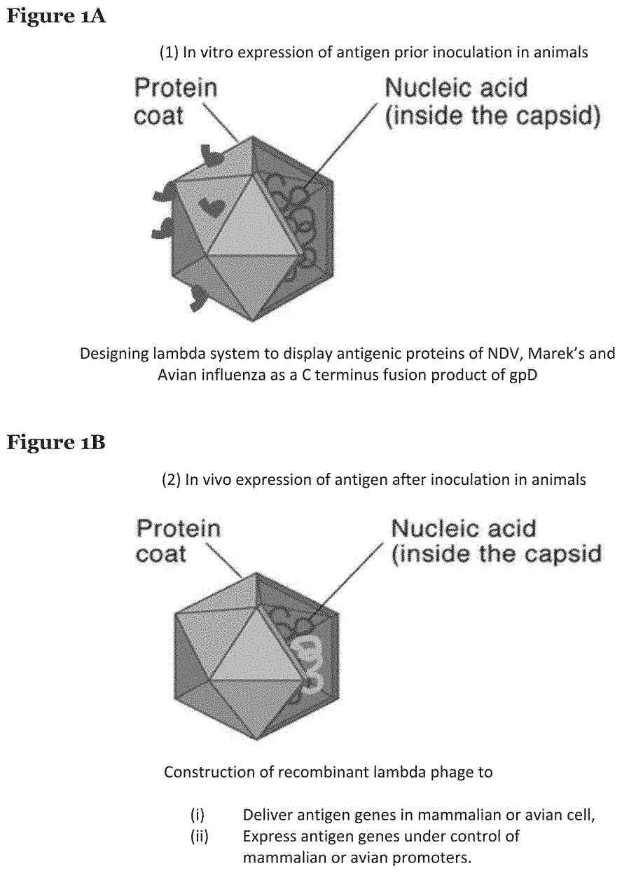

FIG. 1A illustrates a first way in which an icosahedral phage vaccine as described herein can be constructed. Specifically, in this example, a polynucleotide encoding an icosahedral phage head protein, such as the “D” protein of Lambda phage, is used to produce a fusion protein with an antigen of interest. This fusion protein will then be displayed on the icosahedral phage head with multiple copies (up to 405 per phage).

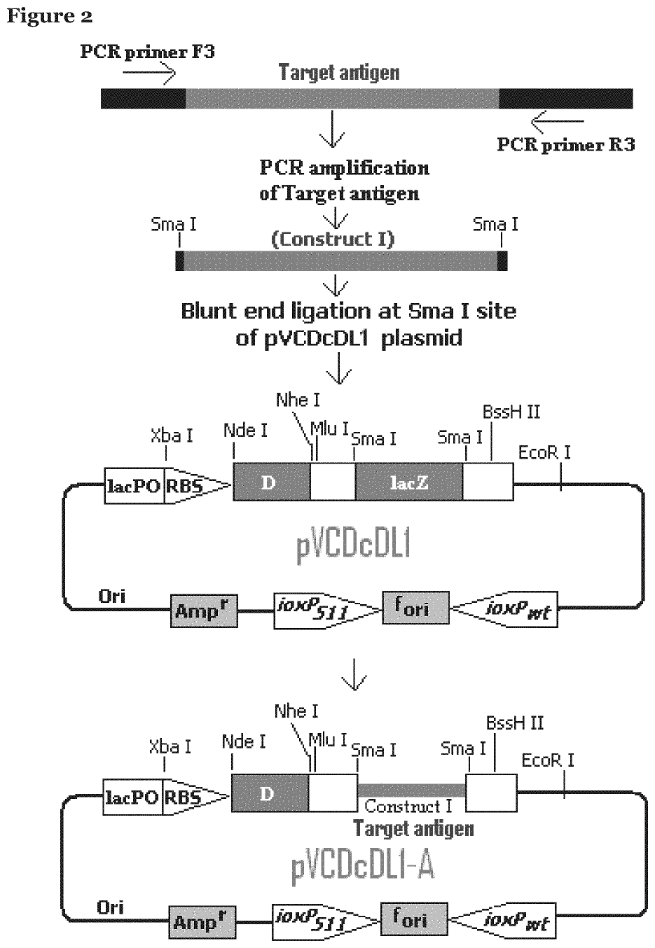

Exemplified constructs that can be used to display the antigen on the phage head are shown in FIG. 2 and are further described in US2007/0207167 (herein incorporated by reference in its entirety.

### Example 2

FIG. 1B illustrates a second way in which an icosahedral phage vaccine as described herein can be constructed. Specifically, an antigen can be delivered as a displayed fusion protein on an icosahedral phage head as described in Example 1. The antigen is fused to the icosahedral phage coat and presented to the hosts' immune cells via a transdermal patch as described herein.

Additionally, the icosahedral phage vaccine can also comprise a polynucleotide inserted into the icosahedral phage genome. In this embodiment, the polynucleotide comprises a nucleotide sequence encoding at least one antigen, wherein the nucleotide sequence is operably associated with promoter capable of being expressed in a mammalian cell.

Here, the polynucleotide is integrated into the icosahedral phage genome—preferably in the beta region of the icosahedral phage, such as in Lambda phage, as this region does not appear to be expressed in bacteria. In this way the polynucleotide will express the encoded antigen when taken up by a mammalian cells, such as for example, mammalian dendritic cells.

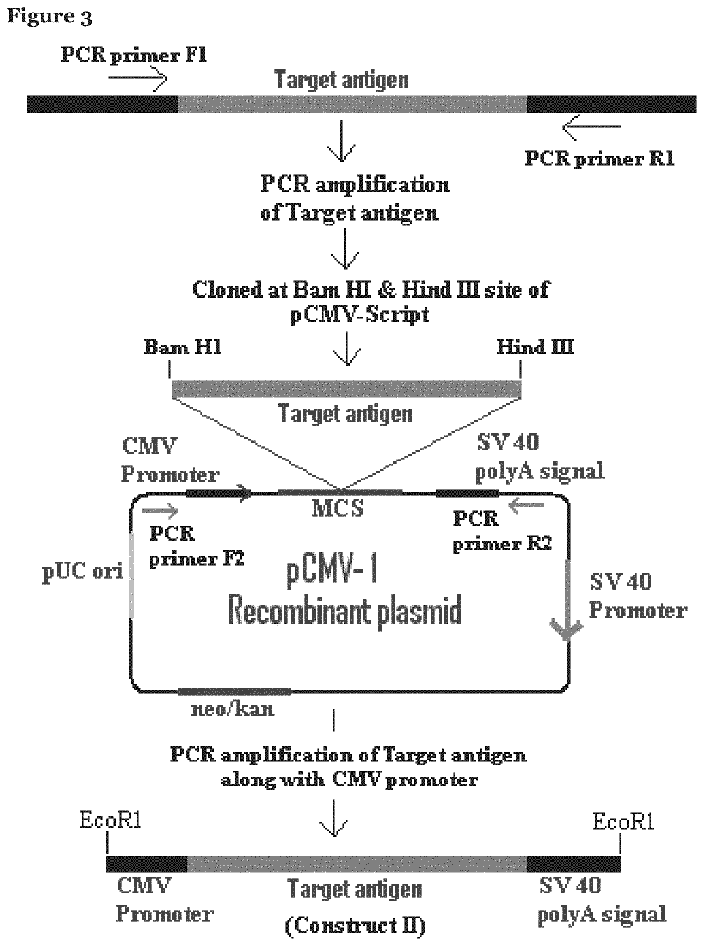

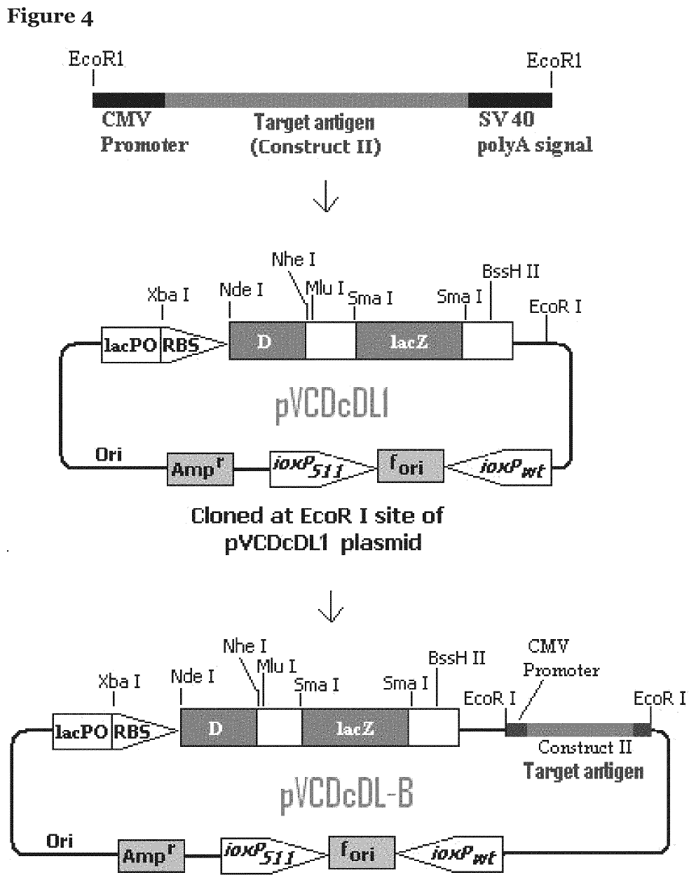

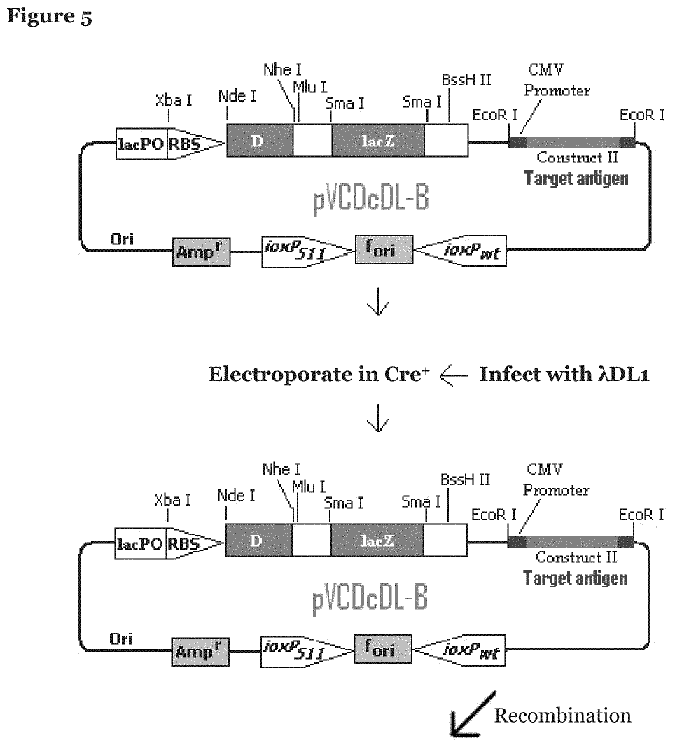

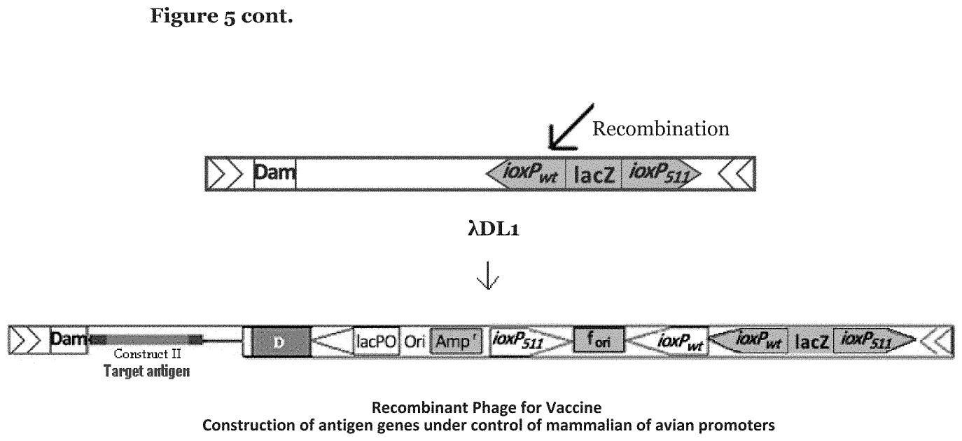

Exemplified constructs that can be used to integrate at least one antigen in the icosahedral phage vaccine genome are shown in FIGS. 3-5 and are further described in US2007/0207167 (herein incorporated by reference in its entirety).

Modifications of the above embodiments, further embodiments and modifications thereof will be apparent to the skilled person on reading this disclosure, and as such these are within the scope of the present invention.

All documents and sequence database entries (if applicable) mentioned in this specification are incorporated herein by reference in their entirety for all purposes.

### REFERENCES

1. Glenn G M, 7. Kenney R T. Mass vaccination: solutions in the skin. Curr Top Microbiol Immunol 2006; 304:247-268. [PubMed: 16989274]
2. Weniger, B G.; Papania, M. Vaccines. Vol. Edn. 5th. Plotkn, S.; Orenstein, W.; Offit, P., editors. Philadelphia: Elsevier; 2008. p. 1357-1392.
3. Miller M A, Pisani E. The cost of unsafe injections. Bull World Health Organ 1999; 77:808-811. [PubMed: 10593028]
4. Zhao 39. Y L, et al. Induction of cytotoxic T-lymphocytes by electroporation-enhanced needle-free skin immunization. Vaccine 2006; 24:1282-1290. [PubMed: 16225969]
5. Ogura M, Paliwal S, Mitragotri S. Low-frequency sonophoresis: Current status and future prospects. Adv Drug Deliv Rev. 2008 61.
6. http://www.3m.com/3M/en_US/drug-delivery-systems-us/technologies/transdermal/components/; http://www.3m.com/3M/en_US/drug-delivery-systems-us/technologies/transdermal/7.
7. Biswas et al., U.S. Patent Application 20140271689/International Patent Application WO2014/145498 A2 (2014)
8. Chaudhary et al., U.S. Pat. No. 7,410,801 (2008).
9. Prausnitz et al., Nat. Biotechnol. (2008) 26(11): 1261-1268.

What is claimed:

1. A transdermal membrane comprising a non-infectious icosahedral phage vaccine displaying at least one antigen, wherein the membrane is stable at room temperature for greater than 3 months.

2. The membrane of claim 1, wherein the non-infectious icosahedral phage vaccine is heat inactivated.

3. The membrane of claim 1, wherein the non-infectious icosahedral phage vaccine is inactivated using UV light.

4. The membrane of claim 1, wherein the non-infectious icosahedral phage vaccine is inactivated prior to application onto the membrane.

5. The membrane of claim 1, wherein the non-infectious icosahedral phage vaccine is inactivated after application onto the membrane.

6. The membrane of claim 1, wherein the membrane is stable at room temperature for greater than 6 months, 9 months, 12 months, 18 months, 24 months, 30 months or 36 months.

7. The membrane of claim 1, wherein the membrane is capable of abrading the skin surface.

8. The membrane of claim 1, wherein the antigen is displayed as a fusion protein with an icosahedral phage coat protein.

9. The membrane of claim 8, wherein the icosahedral phage coat protein is selected from D major coat protein.

10. The membrane of claim 8, wherein the antigen is selected from:
(a) a bacterium or a cancer antigen;
(b) a cancer antigen selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p1800erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68 \KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, mesothelin or any combination thereof;
(c) a bacterium selected from a Risk Group IV bacterium;
(d) a Risk Group IV bacterium selected from Arenaviruses (e.g., Guanarito virus, Lassa virus, Junin virus, Machupo virus, Sabia, Bunyaviruses (Nairovirus): Crimean-Congo hemorrhagic fever virus), Filoviruses (e.g., Ebola virus and Marburg virus), Flaviruses (Togaviruses)(e.g., Group B Arboviruses: Tick-borne encephalitis virus complex including Absetterov, Central European encephalitis, Hanzalova, Hypr, Kumlinge, Kyasanur Forest disease, Omsk hemorrhagic fever, and Russian spring-summer encephalitis viruses), Herpesviruses (alpha) (Herpesvirus simiae (Herpes B or Monkey B virus)), Paramyxoviruses (e.g., Equine morbillivirus (Hendra virus)); Hemorrhagic fever agents and viruses as yet undefined, or any combination thereof.

11. The membrane of claim 10, wherein the Hemorrhagic fever agent is selected from Ebolavirus, Bundibugyo ebolavirus, Reston ebolavirus, Sudan ebolavirus, Taï Forest ebolavirus (originally Côte d'Ivoire ebolavirus), Zaire ebolavirus, or any combination thereof.

12. The membrane of claim 1, wherein more than one antigen is displayed.

13. The membrane of claim 1, wherein the icosahedral phage vaccine further comprises a polynucleotide encoding a second antigen operably associated with a promoter capable of being expressed in a mammalian cell.

14. The membrane of claim 13, wherein the second antigen is derived from the same protein as the displayed antigen.

15. The membrane of claim 13, wherein the second antigen is different from the displayed antigen.

16. The membrane of claim 13, wherein the polynucleotide is inserted into the icosahedral phage vaccine genome.

17. The membrane of claim 1, wherein the polynucleotide encodes for multiple antigens.

18. The membrane of claim 13, wherein the second antigen is selected from:
(a) a bacterium or a cancer antigen;
(b) a cancer antigen selected from: MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, GAGE-I, GAGE-2, GAGE-3, GAGE-4, GAGE-5, GAGE-6, GAGE-7, GAGE-8, BAGE-I, RAGE-1, LB33/MUM-1, PRAME, NAG, MAGE-Xp2 (MAGE-B2), MAGE-Xp3 (MAGE-B3), MAGE-Xp4 (MAGE-B4), MAGE-C1/CT7, MAGE-C2, NY-ESO-I, LAGE-I, SSX-I, SSX-2(HOM-MEL-40), SSX-3, SSX-4, SSX-5, SCP-I and XAGE, melanocyte differentiation antigens, p53, ras, CEA, MUC1, PMSA, PSA, tyrosinase, Melan-A, MART-1, gp100, gp75, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-2, and 3, neo-PAP, myosin class I, OS-9, pml-RAR alpha fusion protein, PTPRK, K-ras, N-ras, Triosephosphate isomerase, GnTV, Herv-K-mel, NA-88, SP17, and TRP2-Int2, (MART-I), E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, alpha.-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, TPS, tyrosinase related proteins, TRP-1, TRP-2, mesothelin or any combination thereof;
(c) a bacterium selected from a Risk Group IV bacterium;
(d) a Risk Group IV bacterium selected from Arenaviruses (e.g., Guanarito virus, Lassa virus, Junin virus, Machupo virus, Sabia, Bunyaviruses (Nairovirus): Crimean-Congo hemorrhagic fever virus), Filoviruses (e.g., Ebola virus and Marburg virus), Flaviruses (Togaviruses)(e.g., Group B Arboviruses: Tick-borne encephalitis virus complex including Absetterov, Central European encephalitis, Hanzalova, Hypr, Kumlinge, Kyasanur Forest disease, Omsk hemorrhagic fever, and Russian spring-summer encephalitis viruses), Herpesviruses (alpha) (Herpesvirus simiae (Herpes B or Monkey B virus)), Paramyxoviruses (e.g., Equine morbillivirus (Hendra virus)); Hemorrhagic fever agents and viruses as yet undefined, or any combination thereof.

19. The membrane of claim 18, wherein the Hemorrhagic fever agent is selected from Ebolavirus, Bundibugyo ebolavirus, Reston ebolavirus, Sudan ebolavirus, Taï Forest ebolavirus (originally Côte d'Ivoire ebolavirus), Zaire ebolavirus, or any combination thereof.

20. A method of vaccination a subject in need thereof, wherein the method comprises contacting the skin of the subject with the membrane of claim 1.

21. The method of claim 20, wherein the subject is a human.

22. The method of claim 20, wherein the subject is a non-human.

23. The method of claim 20, wherein the subject is being vaccinated against cancer or a bacterial infection.

24. The method of claim 23, wherein the cancer is selected from: a sarcoma, skin cancer, melanoma, bladder cancer, brain cancer, breast cancer, uterus cancer, ovary cancer, prostate cancer, lung cancer, colorectal cancer, cervical cancer, liver cancer, head and neck cancer, esophageal cancer, pancreas cancer, renal cancer, stomach cancer, multiple myeloma, cerebral cancer, adenocarcinoma, pancreatic cancer, or pancreatic ductal adenocarcinoma.

25. The method of claim 23, wherein the bacterial infection is selected from an infection caused by a Risk Group IV bacterium.

26. The method of claim 25, wherein the Risk Group IV infection is a hemorrhagic infection.

27. The method of claim 20, wherein the method is:
(a) performed prophylactically; and/or
(b) repeated to boost the immune response; and/or
(c) part of a prime-boost protocol.
